(19) United States
(12) Patent Application Publication
(10) Pub. No.: US 2006/0217729 A1
(43) Pub. Date: Sep. 28, 2006

(54) Surgical apparatus and tools for same

(75) Inventors: Dana Allen Eskridge, Ojai, CA (US); Dennis L. Foster, Ojai, CA (US); Douglas William Haight, Camarillo, CA (US); Laura A. Kriese, Ventura, CA (US)

Correspondence Address: FITZPATRICK CELLA HARPER & SCINTO, 30 ROCKEFELLER PLAZA, NEW YORK, NY, 10112, US

(73) Assignee: Brasseler USA Medical LLC, Savannah, GA (US)

(21) Appl. No.: 11/074,679

(22) Filed: Mar. 9, 2005

(51) Int. Cl. A61B 17/00

(52) U.S. Cl. 606/80

(57) ABSTRACT

A surgical power tool includes a hand unit and a tool holder. The tool holder engages a hub of a tool to hold the tool during use. One tool holder includes a substantially planar support surface, a pair of spaced-apart sidewalls protruding from the support surface, each sidewall having at least one tab extending from the sidewall toward the other of the sidewalls. A tool clamp, including a post protrudes from the support surface with an enlarged head at a distal end thereof. A lock button protrudes from the support surface, the lock button being depressible toward the support surface. Another tool holder includes an output shaft having a driven end and a free end, the free end of the output shaft having a slot formed therein. A collet is disposed coaxially with the output shaft and is rotatable relative thereto. The collet has a collet wall extending between the first and second ends of the collet. The collet has a plurality of slots in the first end of the collet, a plurality of grooves in an inner surface of the collet wall, and a plurality of internal ridges protruding from the inner surface of the collet wall.

### FIELD OF THE INVENTION

Our invention relates generally to surgical apparatuses, and tools and power modules for the same, and to a method of preparing a surgical apparatus. More specifically, our invention relates to surgical apparatuses having tool holders that facilitate insertion of a tool, to tools having hubs that facilitate insertion of the tools in tool holders, to power modules having a power supply and an electric motor for use in surgical apparatuses, and to a method of preparing such a surgical apparatus for surgery.

### BACKGROUND OF THE INVENTION

Orthopedic surgery requires a great degree of precision on the part of the surgeon. At the same time, orthopedic surgery often requires the removal or alteration of hard and/or tough materials, such as bone and tissue. Thus, orthopedic surgical tools must be sufficiently powerful and durable to cut or otherwise alter bone and tissue, while at the same time offering the degree of precision and control necessary to perform the often-delicate surgical procedures.

Powered surgical apparatuses are known in the art for use in orthopedic procedures. Such powered apparatuses are typically pneumatic or battery powered, and may be adapted for various orthopedic procedures such as drilling, screwing, reaming, wire driving, pinning and sawing (both reciprocating and sagittal varieties). Depending on the particular orthopedic procedure, the powered apparatuses may be equipped with one or more tools, such as saw blades, drills, driver bits, reams, wire driving or pinning attachments, and the like.

While improvements have been made to powered surgical apparatuses in recent years, various drawbacks still remain. One drawback is that many powered surgical apparatuses require at least two hands to install and/or change the tool attached to the apparatus. For example, U.S. Pat. No. 5,439,472 describes a surgical tool chuck, which requires two hands for insertion of the blade. However, during a surgical procedure, the surgeon may not always have two hands free to insert and/or change the tool. U.S. Pat. Nos. 5,697,158 and 5,839,196 describe other conventional powered surgical apparatuses requiring two or more hands for insertion of a tool.

Another drawback is that, in order to securely attach tools, existing powered surgical apparatuses typically employ a tool holder having a slot or other enclosure for insertion of the tool. Such enclosures tend to have blind crevices, which are difficult to clean thoroughly. In addition, such enclosures may obstruct the surgeon's view of the surgical sight during use.

Still another drawback of some existing surgical apparatuses is that their tool holders do not securely hold and seat the tool. For example, U.S. Pat. No. 4,020,555 describes a connecting mechanism for a reciprocating saw blade, in which a collar is spring biased to a locking position. The collar can be rotated to a position where slots in the collar align with slots in a reciprocating shaft for insertion or removal of a blade. The blade is locked in place by releasing the collar. However, in this arrangement, the blade may not be securely held or seated in the tool holder.

A drawback associated with some conventional tools is that, when the tool is held in a tool holder of a surgical apparatus, there is a clearance between the lateral edges of the tool and the tool holder. This clearance results in inefficiencies, such as reduction in movement of the working end of a tool. U.S. Design Pat. Nos. Des. 337,160 and Des. 385,163 show tools that suffer from this drawback.

Another drawback associated with electrically powered surgical apparatuses is that they are prone to damage during sterilization of the surgical apparatus. It is standard practice in the surgical environment to sterilize electrically powered devices used in the operating room using a steam sterilization process prior to their use. The sterilization process subjects the electrical components to saturated steam and extremely high temperatures. Such hostile conditions lead to premature failure of the electronic components. These electronic failures are an ongoing source of frustration in the surgical environment.

In an attempt to minimize these failures, so-called “sterile transfer design” electrically powered surgical apparatuses have been developed that do not require the battery pack to be sterilized prior to use. For example, U.S. Pat. No. 4,091,880 discloses a portable surgical wire inserting instrument, the housing handle of which holds a removable power pack in which is mounted a motor and a source of power electrically connected to the motor. In addition, U.S. Pat. No. 5,957,945 discloses a powered handpiece, having a handpiece body which is capable of being sterilized to medical standards prior to each use, and a motor assembly which is non-sterile. The motor assembly is removable from the handpiece prior to sterilization and can be reinstalled in the sterilized handpiece body without contaminating the handpiece body, by using a reusable funnel. However, even in these sterile transfer design devices, other electronic components of the device, such as an electronic controller, sensors, switches, and the like, are still subjected to damaging steam sterilization.

### SUMMARY OF THE INVENTION

Our invention remedies these and other drawbacks of the existing powered surgical apparatuses, and provides a surgical apparatus that is easy to use, easy to clean, and is not susceptible to damage from sterilization. Our invention also applies to tools and power modules for use with such a surgical apparatus, and to a method of preparing such a surgical apparatus for surgery.

In one aspect, our invention relates to a surgical apparatus comprising a tool holder. The tool holder comprises substantially planar support surface, with a pair of spaced-apart sidewalls protruding from the support surface. Each sidewall has at least one tab extending from the sidewall toward the other of the sidewalls. A tool clamp is provided, including a post protruding from the support surface with an enlarged head at a distal end thereof. The tool clamp is preferably spaced substantially equally from each of the sidewalls. A lock button protrudes from the support surface, and is depressible toward the support surface. The lock button is also preferably spaced substantially equally from each of the sidewalls. Preferably, the tool holder further comprises a boss protruding from the support surface and being spaced substantially equally from each of the sidewalls. More preferably, the boss has a peripheral wall that is substantially perpendicular to the support surface, and has a substantially wedge-shaped perimeter adapted to engage surfaces of a tool hub to minimize a lateral clearance between the tool hub and the sidewalls of the tool holder. Also, the tool clamp is preferably slidable in an aperture in the support surface, and is biased toward the support surface to provide a clamping force for clamping a tool.

In another aspect, our invention relates to a tool comprising an elongated body having a working surface disposed at a first end of the elongated body, and a hub, by which the tool can be held, disposed at a second end of the elongated body. The hub comprises a pair of lateral side surfaces and a slot having an opening at the second end of said elongated body. A terminus of the slot is spaced from the opening of the slot toward the first end of the elongated body. A pair of substantially parallel slot walls extends from he opening to the terminus. The slot has an expanded portion located between the opening and the terminus. Preferably, each lateral side surface has a notch formed therein. Also preferably, the expanded portion comprises a pair of arcuate notches, one notch being formed on each of the slot walls. Preferably the hub further comprises a pair of stiffening surfaces, one stiffening surface extending between each of the slot walls and the second end of the elongated body, such that the stiffening surfaces are angled with respect to both the slot walls and the second end of said elongated body. The stiffening surfaces are adapted to engage a boss of a tool holder.

In another aspect, our invention relates to a surgical apparatus comprising a tool holder. The tool holder comprises an output shaft having a driven end and a free end, the free end of the output shaft having a slot formed therein. A collet is disposed coaxially with the output shaft and is rotatable relative thereto. The collet has a first end adjacent to the free end of the output shaft, a second end spaced from the free end of the output shaft toward the driven end of the output shaft, and a collet wall extending between the first and second ends of the collet. The collet has a plurality of slots in the first end of the collet, a plurality of grooves in an inner surface of the collet wall, and a plurality of internal ridges protruding from the inner surface of the collet wall. Preferably, the tool holder further comprises a section ring disposed in an annular groove formed in an outer surface of the output shaft.

In still another aspect, our invention relates to a tool comprising an elongated body having a first end, a second end, and two lateral edges. A working surface is disposed along at least a portion of one lateral edge near the first end of the elongated body. A hub, by which the tool can be held, is disposed at the second end of the elongated body. The hub comprises a middle portion having a first width. A pair of lateral recesses is formed in the hub adjacent to the middle portion in the direction of the first end of the elongated body. A pair of tangs protrude laterally from the hub adjacent to the lateral recesses in the direction of the first end of the elongated body. The hub also includes a narrow end portion, having a width less than the first width, adjacent to the middle portion at an end of said hub opposite the working surface.

In another aspect, our invention relates to a surgical apparatus comprising a hand unit and a detachable power module coupled to the hand unit for providing power to the hand unit. The power module comprises a housing, an electric motor in the housing to provide mechanical energy to the hand unit, an electrical power supply in the housing to provide electrical energy to the electric motor; and an electronic controller in the housing for controlling the electric motor. The power module may be non-sterile. Preferably, the electronic controller comprises a programmable electronic controller, which is capable of storing a plurality of programs for monitoring and controlling functions of the power module.

In another aspect, our invention relates to a power module for use with a surgical apparatus. The power module comprises a housing, an electric motor in the housing to provide mechanical energy to the hand unit, an electrical power supply in the housing to provide electrical energy to the electric motor; and an electronic controller in the housing for controlling the electric motor. Preferably, the electronic controller comprises a programmable electronic controller, which is capable of storing a plurality of programs for monitoring and controlling functions of the power module.

In yet another aspect, our invention relates to a method of preparing a surgical apparatus for surgery. The method comprises (a) sterilizing a hand unit, (b) inserting a power module comprising an electric motor, a power supply, and an electronic controller in the receptacle in the hand unit, and (c) sealing the power module inside the receptacle in the hand unit. Preferably the method further comprises the steps of (d) providing a sterile transfer sleeve, (e) installing the sterile transfer sleeve on an opening of the receptacle of the hand unit prior to inserting the power module in the receptacle of the hand unit in step (b), and (f) removing the transfer sleeve after the power module has been installed in step (b) and prior to sealing the receptacle in step (c).

The tools and power modules described herein may be advantageously used with one or more of the surgical apparatuses described herein. Moreover, the surgical apparatuses and power modules described herein can be used to practice the method of our invention.

A better understanding of these and other features and advantages of our invention may be had by reference to the drawings and to the accompanying description, in which preferred embodiments of the invention are illustrated and described.

### BRIEF DESCRIPTION OF THE DRAWINGS

FIG. 1 is a perspective view of a surgical apparatus according to one preferred embodiment of our invention.

FIG. 2 is a cross-sectional view of the surgical apparatus of FIG. 1, taken along line 2-2 in FIG. 1.

FIG. 2A is a cross-sectional view of the surgical apparatus of FIG. 1, taken along line 2A-2A in FIG. 1.

FIG. 2B is a partial left side view of the surgical apparatus of FIG. 1.

FIG. 2C is a partial cross-sectional view of the surgical apparatus of FIG. 1, taken along line 2C-2C in FIG. 2B.

FIG. 3 is a perspective view of the tool holder of the surgical apparatus of FIG. 1.

FIG. 4 is plan view of a tool usable with the surgical apparatus of FIG. 1.

FIG. 5 is a partial perspective view, showing the tool of FIG. 4 installed in the tool holder of FIG. 3.

FIGS. 5A, 5B, and 5C are detail views of the tool holder of the surgical apparatus of FIG. 1, taken along line 5A-5A in FIG. 5, and show a tool at three different stages of insertion in the tool holder.

FIG. 6 is a cross-sectional view of a surgical apparatus according to another preferred embodiment of our invention.

FIG. 7 is a perspective, section view of the tool holder of the surgical apparatus of FIG. 6.

FIG. 8 is a plan view of a tool usable with the surgical apparatus of FIG. 6.

FIG. 9 is power module usable with surgical apparatuses according to our invention, with top and side portions of the housing removed for clarity.

FIG. 9A is a perspective view of a sterile transfer introducer sleeve usable with surgical apparatuses according to our invention.

FIG. 10 is flowchart showing a method of preparing a surgical apparatus using a power module according to our invention for surgery

Throughout the figures, like or corresponding reference numerals have been used for like or corresponding parts.

### DETAILED DESCRIPTION OF THE PREFERRED EMBODIMENTS

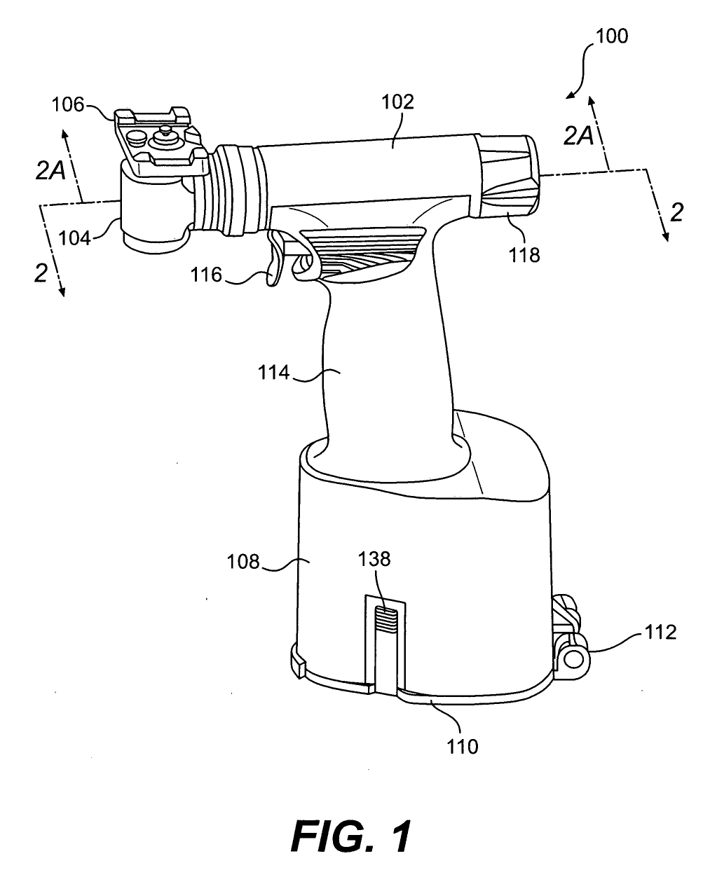

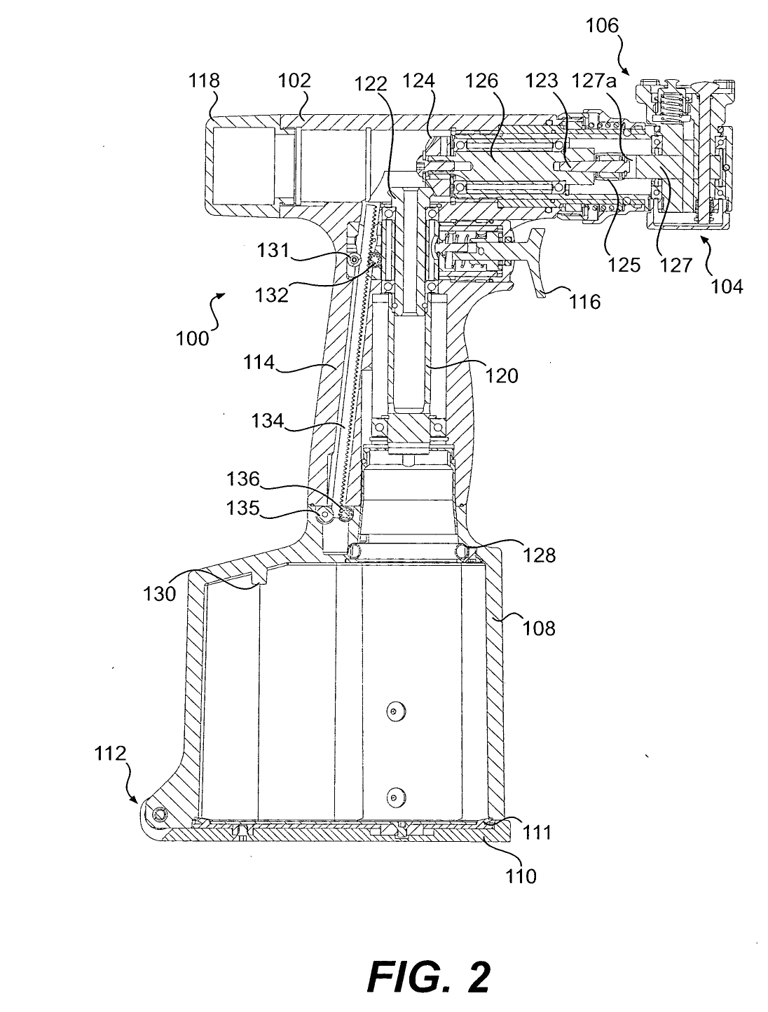

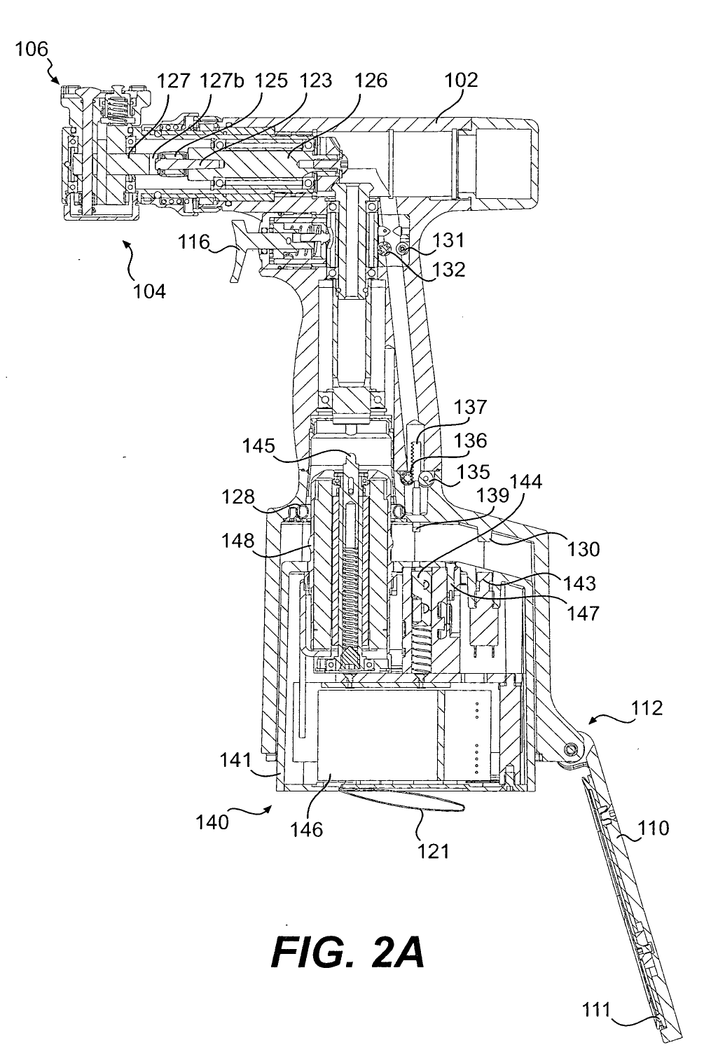

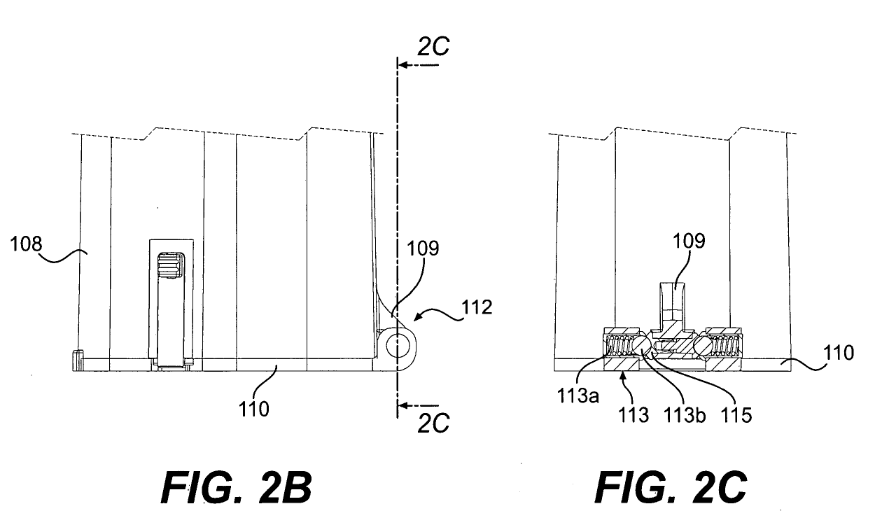

A surgical apparatus 100 according to one preferred embodiment of our invention is illustrated in FIGS. 1 through 5. As shown in FIG. 1, the surgical apparatus 100 generally includes a hand unit 102, an oscillator assembly 104 coupled to the hand unit 102, and, as shown in FIG. 2A, a power module 140 received in a receptacle 108 in the hand unit 102. As described further below, the oscillator assembly 104 rotates a tool holder 106 back-and-forth in an oscillatory manner to move a tool, such as the cutting tool shown in FIG. 4, in an arc. This type of device is generally known as a sagittal saw.

Hand Unit

With reference to FIGS. 1, 2, and 2A, the hand unit 102 is generally gun-shaped, having a handle 114 and a trigger 116 for gripping and actuating the surgical apparatus 100. The receptacle 108 of the hand unit 102 is provided with a lid 110 pivotally attached to the hand unit at a hinge 112. The lid 110 is pivotable between an open position (shown in FIG. 2A) for insertion and removal of the power module 140, and a closed position (shown in FIGS. 1 and 2), in which the power module 140 is sealed within the receptacle 108. The lid 110 can be held in the closed position by a latch 138 and sealed (preferably hermetically) by a seal 111 disposed around the periphery of the receptacle opening. When the latch 138 is released, a support spring 121 causes the lid 110 to partially open. The support spring 121 also holds the power module 140 snugly in the receptacle 108 when the lid 110 is closed, preventing it from rattling around in the receptacle 108. An access cap 118 is provided in the hand unit 102 to allow access to the interior of the hand unit 102 for cleaning and maintenance.

Input to the trigger 116 by a user is preferably communicated to the power module 140 via a series of rack and pinion gears, as shown in FIGS. 2 and 2A. A first rack gear (not shown) is coupled to the trigger 116 by a pin (not shown), and extends and engages a first pinion gear 132 in the hand unit 102, as shown in FIG. 2. As the trigger 116 is depressed, the fist rack gear slides in a groove (also not shown) tangent to the first pinion gear 132, teeth of the first rack gear engaging teeth of the first pinion gear 132 and thereby causing the fist pinion gear 132 to rotate. A second rack gear 134 extends between the first pinion gear 132 and a second pinion gear 136 located near the base of the handle 114. The second rack gear 134 is held in engagement with the first and second pinion gears 132 and 136 by rollers 131 and 135, respectively, and thereby transmits rotation of the first pinion gear 132 to rotation of the second pinion gear 136. As shown in FIG. 2A, a third rack gear 137 also engages the second pinion gear 136, and is held in engagement by the third roller 135. The third rack gear 137 is coupled to an actuation rod 139, which extends into the receptacle 108 of the hand unit 102 and engages a speed control mechanism 144 of the power module 140 to control the speed, direction, and/or oscillation of the motor 142, as discussed in more detail below.

Of course, numerous other mechanical and/or electronic means for communicating user inputs to a trigger or other interface to the power module would be apparent to those of ordinary skill in the art and will, therefore, not be described herein.

Preferably, the trigger 116 also includes a trigger lock feature, whereby the trigger can be rotated to a locked position, so that the surgical apparatus is not accidentally actuated during preparation or handling of the hand unit 102. Various trigger locking features are known in the art, and are within the knowledge of one of ordinary skill in the art.

Preferably, the lid 110 is removable from the hand unit 102 to allow for installation of other types, shapes, and sizes of power modules, or attachment of other modules, such as corded modules, pneumatic modules, and the like. Such a removable lid configuration is shown in FIGS. 2B and 2C, and is accomplished by provision of a breakaway hinge 112. The breakaway hinge 112 comprises a hinge mount 109 formed on a sidewall of the receptacle 108, which engages with a pair of detent mechanisms 113 formed on the lid 110. The detent mechanisms 113, each comprise a ball bearing 113b, which is biased by a spring 113a or other elastomeric member into engagement with concave recesses 115 of the hinge mount 109. Thus, when attached to the hand unit 102, the lid 110 is allowed to pivot about the engagement of the detent mechanisms 113 and the hinge mount 109. To remove the lid 110, the user simply pulls or twists the lid 110 with a force sufficient to overcome the bias of the detent mechanisms 113 and the lid comes free, without the need for any special tools. The lid 110 can be reattached by simply pressing the detent mechanisms 113 back into place on the concave recesses 115 of the hinge mount 109.

As best shown in FIG. 2, the hand unit 102 transfers rotational energy from an electric motor 142 of the power module 140 to oscillatory motion of the tool holder 106 via a drive train in a known manner. In the drive train shown in FIG. 2, an output shaft 145 of the electric motor 142 engages a slotted drive shaft 120 in the hand unit 102. When driven by the electric motor 142, the drive shaft 120 rotates a first bevel gear 122, which in-turn engages and rotates a second bevel gear 124 of the oscillator assembly 104, the first and second bevel gears 122, 124 being oriented such that their axes of rotation are substantially perpendicular to one another. The second bevel gear 124 is coupled to a wobble shaft 126, which has a canted pin 123 protruding from the end of the wobble shaft 126 opposite the second bevel gear 124. That is, a longitudinal axis of the canted pin 123 is at an oblique angle relative to the longitudinal axis of the wobble shaft 126. A bearing 125 is disposed coaxially on the canted pin 123. As the wobble shaft 126 rotates, the bearing 125 on the end of the canted pin 123 moves in a circular path that is coaxial with, but larger in diameter than, the wobble shaft 126. The horizontal components of the path of bearing 125 (into and out of the page in FIG. 2) bring the bearing 125 into oscillatory contact with arms 127a, 127b of a Y-shaped member 127 (only one arm 127a is visible in FIG. 2, the other arm being visible in FIG. 2A). The Y-shaped member 127 is fixed to the tool holder 106. Thus, as the bearing 125 alternately contacts arms 127a, 127b, the Y-shaped member 127 is moved back-and-forth, thereby oscillating the tool holder 106. The foregoing description is of one exemplary drive train that is usable with this embodiment of our invention, and has been provided for completeness. However, the configuration of the drive train does not form a part of our invention, and any conventional drive train could be used to transfer rotational energy from the electric motor 142 to oscillatory motion of the tool holder 106.

When the power module 140 is inserted into the receptacle 108 in the hand unit 102, it is held in place by interaction between a radial ridge 148 on the power module 140 and a cylindrical canted coil spring 128 that resides within the receptacle 108 at the base of handle 114. This arrangement acts as a safety feature to retain the power module 140 in the hand unit 102, and to prevent it from inadvertently falling out of the sterile hand unit 102 into the sterile surgical field. This safety feature is especially important since, as described later, the power module may be non-sterile.

First Tool Holder

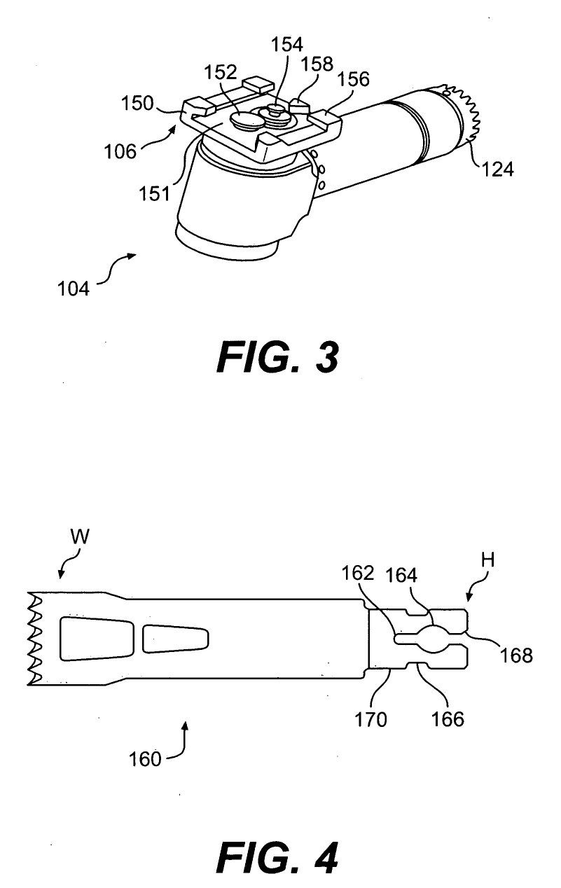

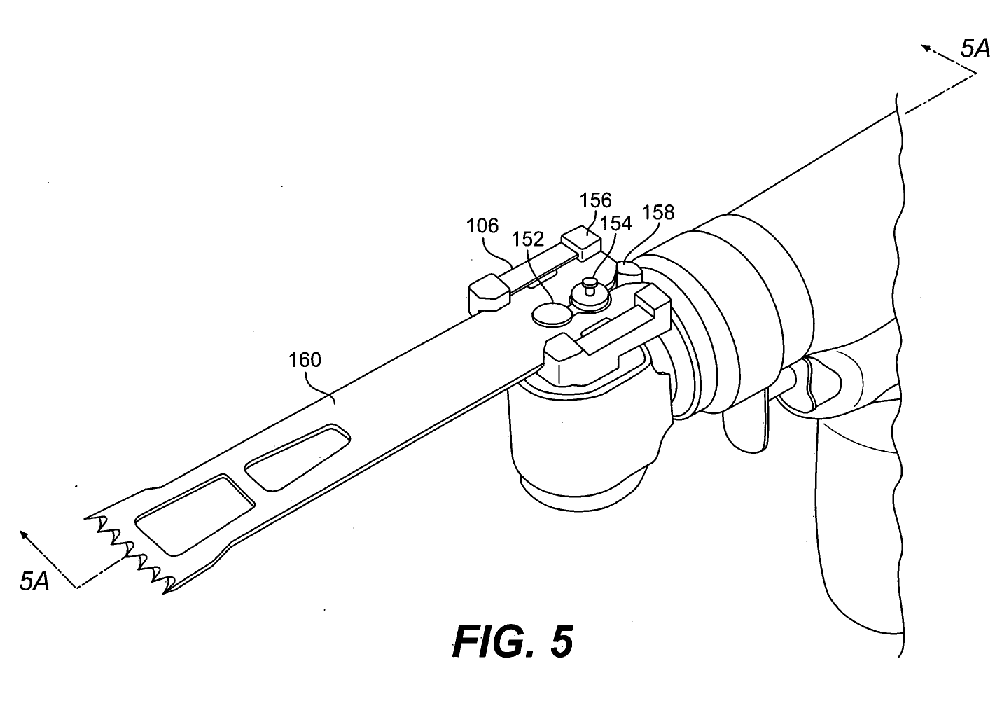

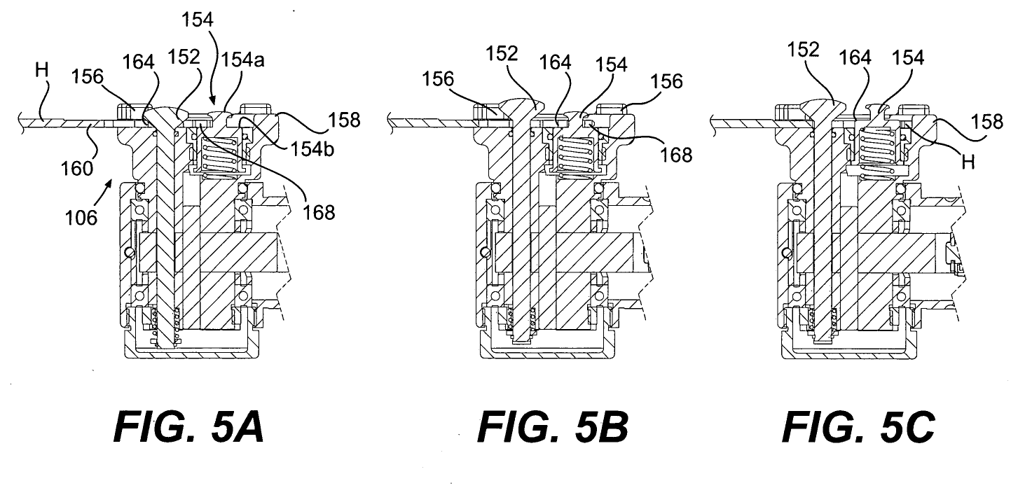

The tool holder 106 of this embodiment will now be described with reference to FIGS. 3 and 5A-5C. The tool holder 106 comprises a substantially planar support surface 151, against which a tool can be held, as a means for supporting the tool. A pair of spaced-apart sidewalls 150 protrude from the support surface 151, each sidewall 150 preferably having a pair of tabs 156 extending from a distal end of the sidewall toward the other of the sidewalls (for a total of four tabs 156). Alternatively, each of the sidewalls 150 may be provided with any number of tabs (one or more), or may be provided with a continuous rail extending along each sidewall. The sidewalls 150 interface with the sides of the tool and constitute means for transferring the driving force to the tool. The four overhanging tabs 156 provide a means for retaining the blade in the head of the saw and, as described below, facilitate one-handed insertion of tools in the tool holder 106. Preferably, the sidewalls 150 protrude substantially perpendicular to the support surface 151 and are substantially parallel to each other. Alternatively, however, the sidewalls 150 may be oriented in other arrangements, such as being slanted toward each other, being slanted with respect to the support surface 151 (e.g., at an angle greater than 90 degrees relative to the support surface 151), being curved, or the like.

A tool clamp 152, comprising a post with an enlarged head at a distal end thereof, protrudes from the support surface 151 and is preferably spaced substantially equally from each of the sidewalls 150. The post of the tool clamp 152 is slidable vertically in an aperture in the support surface 151, and the tool clamp 152 is downwardly biased to provide a clamping means for clamping the tool to the support surface 151. Also, because the tool clamp 152 is downwardly biased, it helps to stabilize the tool and can provide consistent clamping forces over a range of different tool thicknesses. As shown in FIG. 5A, tools having various thicknesses can be inserted beneath the tool clamp 152 due to the beveled under surface of the head of the tool clamp 152.

A lock button 154 protrudes and is biased upwardly from the support surface 151. As described in more detail below, the lock button 154 constitutes locking means for locking the tool in place in the tool holder 106. The lock button 154 is depressible toward the support surface 151 for installation and removal of the tool. As shown in FIG. 5A, the lock button 154 comprises a large round lower platform 154b, from the center of which a thin neck extends up to a smaller upper platform 154a. The lock button 154 is also preferably spaced substantially equally from each of the sidewalls 150. The lock button 154 also is capable of locking tools having various different thicknesses in place in the tool holder 106.

The open design and low profile of this tool holder of our invention allow easy and thorough cleaning of the tool holder after use, and increased visibility of the surgical site during use.

The tool holder 106 also preferably includes a boss 158 protruding from the support surface 151. Preferably, the boss 158 has a peripheral wall substantially perpendicular to the support surface 151, and has a substantially wedge-shaped perimeter. The boss 158 acts as a stiffening means for engaging and stiffening a hub of a tool. As described further below, the wedge-shaped boss 158 interacts with complimentary surfaces on the hub of the tool to spread the hub of the tool and minimize the lateral clearance between the hub of the tool and the sidewalls 151 of the tool holder 106. Alternatively, the boss 158 can be formed in a variety of other shapes, such as rounded, circular, V-shape, and the like. Moreover, the peripheral sidewall of the boss need not be substantially perpendicular to the support surface 151 and may form an angle greater than or less than 90 degrees relative to the support surface 151. In such cases, the stiffening surfaces 168 of the tool hub H may be modified accordingly.

FIG. 4 shows a tool 160 usable with the tool holder 106 of FIG. 3. Preferably, the tool 160 is a cutting tool, such as a sagittal saw blade, having an elongated body, a working surface W (such as a cutting surface for cutting tissue) at a first end of the elongated body, and a hub H by which the tool can be held in the tool holder 106 at the other end of the body. Of course, any type of tool can be provided with a hub H like that of FIG. 4, and thus can be held in tool holder 106 of our invention.

The hub H constitutes hub means for engaging the tool holder and securing the tool. The hub H of the tool comprises a pair of lateral side surfaces 170, each side surface preferably having a notch 166 formed therein. The interaction of the notches 166 and other features of the hub H with the tool holder will be described further below. (The hub may be provided without notches 166, but such notch-less hubs would not be insertable using the one-handed insertion method described herein.) The hub H has a slot 162 therein, the slot having an opening at the second end of the elongated body, a terminus spaced from the opening toward the first end of the elongated body, and a pair of substantially parallel slot walls extending from the opening to the terminus if the slot 162. The slot 162 also has an expanded portion 164 in the slot walls between the opening and the terminus. Preferably, the expanded portion 164 of the slot 162 comprises a pair of arcuate notches, one notch formed on each of the slot walls. Alternatively, the expanded portion 164 may be defined by non-arcuate curves, geometric shapes (e.g., square, hexagonal, octagonal, or the like) or parts of such geometric shapes, and the like.

Also, the hub preferably includes a pair of stiffening surfaces 168, one stiffening surface extending between each of the slot walls and the second end of the elongated body. Thus, the stiffening surfaces 168 are angled with respect to both the slot walls and the second end of the elongated body. The stiffening surfaces 168 may be beveled surfaces, as shown in FIG. 4, or alternatively, they may be curved surfaces (either concave or convex surfaces). As described in more detail below, the stiffening surfaces 168 engage boss 158 of the tool holder 106, thereby minimizing the lateral clearance between the hub H and the tool holder 106 and allowing the hub H to be held more rigidly by the tool holder 106.

The tool 160 can easily be inserted in the tool holder 106 using only one hand. First, the user locates the tool 160 above the tool holder 106, with the notches 166 of the tool positioned directly over the front tabs 156 of the tool holder. Next, as shown in FIG. 5A, the user lowers the tool 160 into contact with the support surface 151. In doing so, the notches 166 and expanded portion 164 of the tool fit over the front set of tabs 156 and the tool clamp 152 of the tool holder, respectively, and the portion of the hub H near the stiffening surfaces 168 presses down on the lower platform 154b of the lock button 154. Thus, as the tool 160 is lowered, the hub H depresses the lock button 154. Next, the user pushes the tool 160 toward the boss 158 of the tool holder 106, as shown in FIG. 5B. As the tool 160 slides toward the boss 158, the post of the tool clamp 152 slides in the tool slot 162 toward the terminus, while the neck of the lock button 154 slides in the slot 162 from the opening toward the expanded portion 164. Meanwhile, the enlarged head of the tool clamp 152 the head of the tool clamp 152 rides up on the top of the hub H and clamps the tool 160 toward the support surface 151, by virtue of the beveled under surface of the head of the tool clamp 152. Once the tool 160 is completely seated, as shown in FIGS. 5 and 5C, the lock button 154 is ejected upward so that the lower platform of the lock button 154 fills the expanded portion 164 of the slot, thereby locking the tool 160 in the tool holder 106. In this locked position, the stiffening surfaces 168 abut the wedge-shaped surface of boss 158, thereby spreading the hub H and minimizing the clearances between the lateral side surfaces 170 of the hub H and the sidewalls 150 of the tool holder 106. The extension of the slot 162 beyond the expanded portion 164, facilitates spreading of the hub H to take up the lateral clearance between the hub H and the sidewalls 150. The stiffening action of this wedge design allows the tool to be held more rigidly, thereby maximizing the cutting effectiveness of the cutting tool 160.

To remove the tool 160, the user merely depresses the lock button 154 and performs the foregoing steps in reverse. Thus, with a tool and tool holder according to this embodiment of our invention, a surgeon or other user can quickly and easily insert and remove tools from a powered surgical apparatus using only one hand. Alternatively, the tool 160 can be removed by depressing the lock button 154, and pulling the tool 160 in the direction of the working surface W of the tool 160.

Second Tool Holder

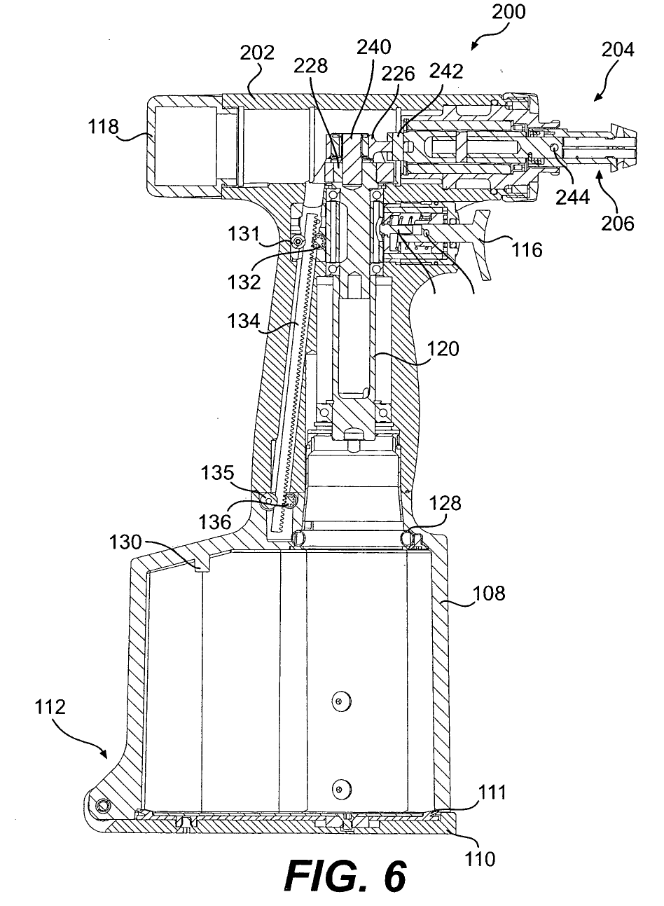

A surgical apparatus 200 according to another embodiment of our invention is illustrated in FIGS. 6 through 8. The surgical apparatus 200 is similar in most respects to the surgical apparatus 100 of the first embodiment. Like reference numerals have been used to identify like features of the second embodiment. For the sake of brevity, a detailed description of these like features has been omitted here. The surgical apparatus 200 of this embodiment differs from the first embodiment in that it includes a reciprocator assembly 204 that drives a collet-style tool holder 206 in a reciprocating motion. This type of surgical apparatus is commonly referred to as a reciprocating or sternum saw. Like the first embodiment, the surgical apparatus 200 of this embodiment includes a hand unit 202. The reciprocator assembly 204 is attached to the hand unit 202. However, the hand unit 202 of this embodiment is different than that of the first embodiment, in that the drive train is adapted in a known manner to engage the reciprocator shaft end 226 of the reciprocator assembly 204.

In this embodiment, the drive shaft 120 is coupled to an eccentric shaft 228, having a pin 240 protruding axially from the end of the eccentric shaft 228 opposite the drive shaft 120. The longitudinal axis of the pin 240 is parallel to, but offset from, the longitudinal axis of the drive shaft 120, such that the pin 240 moves in circular path about the longitudinal axis of the drive shaft 120. The reciprocator shaft end 226 is coupled to the pin 240, and is pivotally connected to the rest of the reciprocator assembly 204 at link pin 242. Thus, the pin 240 moves the reciprocator shaft end 226 in a circular path, the forward and backward components (right and left components in FIG. 6) of which are transmitted to the rest of the reciprocator assembly 204 through the link pin 242, thereby driving the reciprocator assembly 204 in a reciprocating motion. The foregoing description is of one exemplary drive train that is usable with this embodiment of our invention, and has been provided for completeness. However, the configuration of the drive train does not form a part of our invention, and any conventional drive train could be used to transfer rotational energy of from the electric motor 142 to reciprocating motion of the tool holder 206.

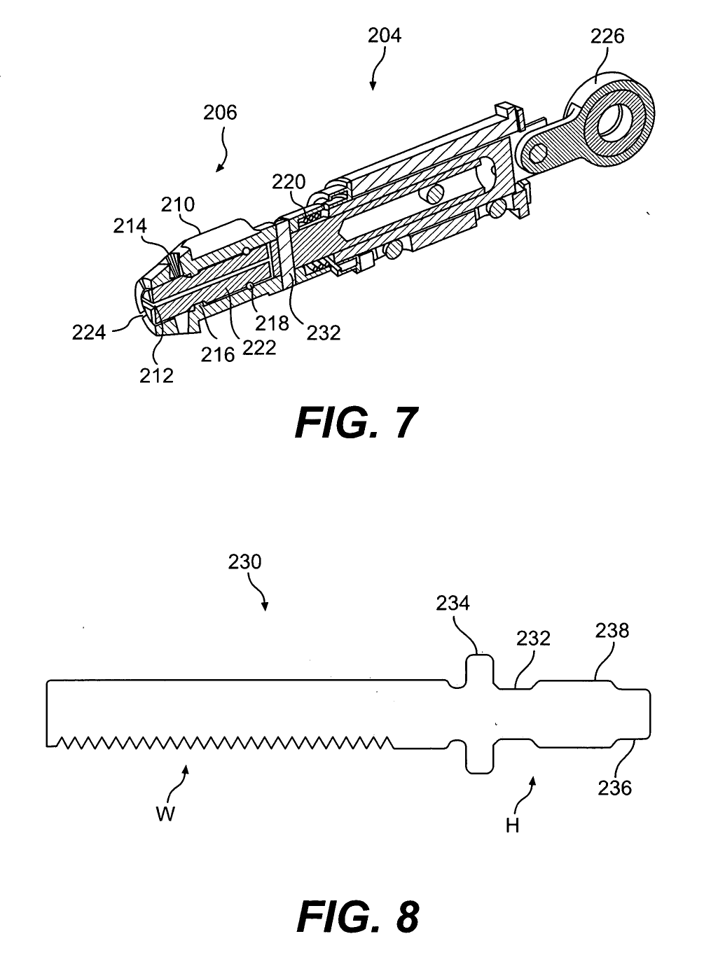

The tool holder 206 of this embodiment will now be described with reference to FIG. 7. The tool holder 206 comprises an output shaft 222 having a driven end and a free end, the free end having one or more slots 212 that bisects the output shaft 222 down its axis for a portion of its distance. The output shaft 222 constitutes holding means for receiving and holding a tool. A collet 210 is disposed on the output shaft 222 and is rotatable relative thereto. The collet 210 and the output shaft 222 are coaxial, with the inner wall of the collet 210 being adjacent to the outer surface of the output shaft 222. One end (the first end) of the collet 210 is adjacent to the free end of the output shaft 222, with the other end (the second end) of the collet 210 being spaced toward the driven end of the output shaft 222.

A plurality of slots 224 is formed in the first end of the collet 210. One or more of the slots 224 in the collet can be aligned with one or more of the slots 212 in the output shaft 222 by rotating the collet 210 relative to the output shaft 222. When aligned, a tool can be inserted in and/or removed from slots 212, 224 of the tool holder 206. A rotation-limiting pin 244 extends through the output shaft 222 and fits in partial circumferential paths (not shown) formed in the collet 210. The rotation-limiting pin 244 stops the collet when the slots 224 of the collet are aligned with the slots 212 in the output shaft 222.

A plurality of spiral grooves 214 is formed in the inner surface of the collet wall. The term “spiral” refers to the fact that the grooves 214 extend in paths having both circumferential and axial components (similar to the spiral grooves in a drill bit). The grooves 214 constitute seating means for pulling the tool into a seated position in the output shaft 222. Also, a plurality of internal ridges 216 protrudes from the inner surface of the collet wall and act as a secondary securing feature to ensure that the tool hub H is securely held in the tool holder 206. The plurality of internal ridges 216 may extend in paths substantially parallel to those of the plurality of grooves 214, or they may have substantially circumferential paths. The internal ridges 216 constitute securing means for retaining the tool in the tool holder 206.

Preferably, the tool holder 206 includes a torsion spring 220 that biases the collet 210 for rotation toward a lock position (shown in FIG. 7). The torsion spring 220 constitutes locking means for locking the tool in output shaft 222 during use. Of course, other types of springs or elastic members could alternatively be used apply a rotational bias to the collet 210. When a tool is installed, the rotational bias works in combination with the spiral grooves 214 to pull the tool into a fully seated position. The tool holder 206 also preferably includes a section ring 218 disposed in an annular groove formed in an outer surface of the output shaft 222. The round section ring 218 effectively reduces the width of the slots 212 in the output shaft 222. In this manner, the section ring 218 serves as a tool hub identifier to assure that only certain tools, having hubs that fit within the section ring 218, can be inserted and locked into the tool holder 206. This safety feature prevents the user from accidentally inserting a tool of the wrong type. The section ring 218 also acts as a locating means for locating the hub of the tool within the output shaft 222.

FIG. 8 shows a tool 230 usable with the tool holder 206 of FIG. 7. Preferably, the tool 230 is a cutting tool, such as a reciprocating saw blade, having an elongated body, a working surface W (such as a cutting surface for cutting tissue) along at least one lateral edge, and a hub H by which the tool can be held in the tool holder 206. Of course, any type of tool can be provided with a hub H like that of FIG. 8, and thus can be held in tool holder 206 of our invention.

The hub H constitutes hub means for engaging the tool holder 206 and securing the tool thereto. The hub H of the tool 230 of this embodiment includes a middle portion 238 having a first width. A pair of lateral recesses 232 is formed in the hub H adjacent to the middle portion 238 in the direction of the working surface W (the first end), and a pair of tangs 234 protrudes laterally from the hub H adjacent to the lateral recesses 232 in the direction of the first end. The hub H also preferably includes a narrow end portion 236, having a width less than the first width. The narrow portion 236 is located adjacent to the middle portion 238 at the end (the second end) of the hub H opposite the working surface W, and is intended to act as a keying feature with the round section ring 218 on the output shaft 222.

The tool 230 can easily be inserted in the tool holder 206. First, the user rotates the collet 210 to align the slots 224 in the collet with the slots 212 in the output shaft. Next, the user inserts the hub H of a tool 230 into the aligned slots 212, 224. At this point, the narrow end portion 236 of the hub H will be received and located by the section ring 218, the tangs 234 of the hub H will be aligned with the spiral grooves 214 of the collet, and the lateral recesses 232 of the hub H will be aligned with the internal ridges 216 of the collet. Next, to secure the tool 230 in the tool holder 206, all the user has to do is release the collet 210. The rotational bias from the torsion spring 220 then rotates the collet 210 into a lock position. At the same time, the grooves 214 within the collet 210 receive the tangs 234 of the hub H and, due to their spiral paths, pull the tool 230 into a fully seated position, thereby stabilizing the tool. The engagement of the grooves 214 with the tangs 234 creates the primary means of locking the tool 230 into the tool holder 206. Also at the same time, the internal ridges 216 in the collet 210 interact with the lateral recesses 232 on the hub H, and provide a secondary blade-locking feature. In this manner, a tool can easily be inserted in the tool holder 206, and held securely in place.

Power Module

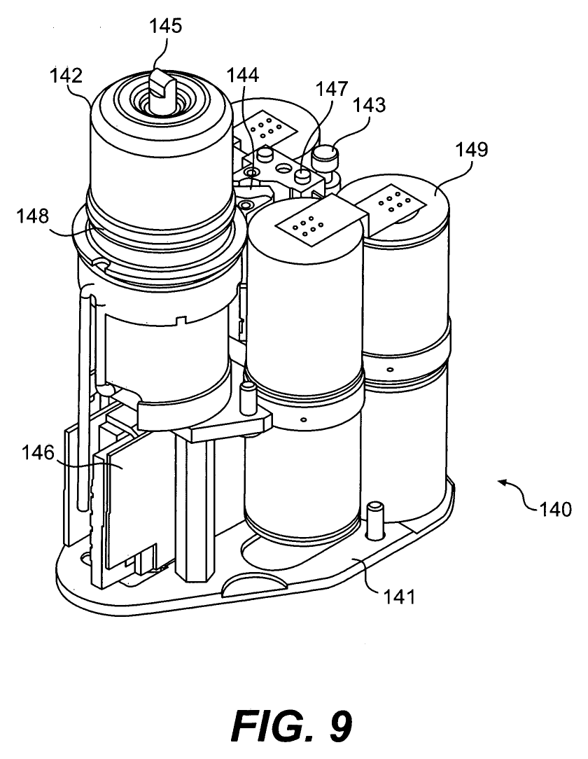

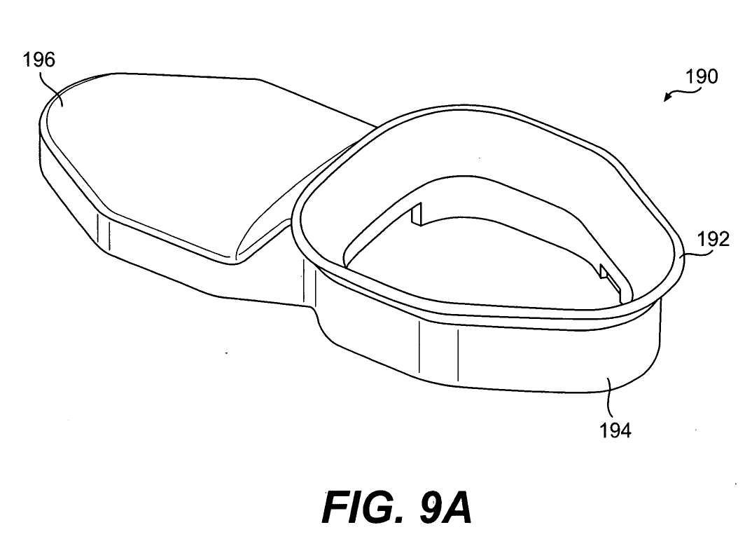

A power module 140 usable with both of the foregoing embodiments, as well as other powered surgical apparatuses, is illustrated in FIGS. 2 and 9. For simplicity, the power module 140 will be described as applied to the first embodiment. Generally, the power module 140 is a self-contained drive unit that can be installed into various powered surgical apparatuses, such as saws and drills. The power module 140 contains substantially all of the electrical components of the surgical apparatus in a single unit. Because the power module 140 is completely enclosed and sealed within the hand unit 102, the power module 140 need not be sterile and, accordingly, need not be subjected to steam sterilization. Thus, the power module 140 overcomes the problems of conventional apparatuses related to electronic failures caused by exposure to moisture and extreme heat during steam sterilization. However, sterilizable power modules may also be used with our invention. In that case, the power module need not be sealed within the receptacle 108 and may or may not protrude from the receptacle. If need be, the lid 110 may be removed to accommodate power modules of various shapes and sizes.

As shown in FIG. 2, the power module 140 has a housing 141. The side and top portions of the housing 141 are not shown in FIG. 9, for clarity. Referring to that figure, the power module 140 also includes an electric motor 142 as a drive means for providing mechanical energy to drive the hand unit 102, rechargeable batteries 149 as electrical power supply means for providing electrical energy to the motor 142 and other electrical components of the power module 140, and an electronic controller 146 as control means for controlling the electric motor.

The electric motor 142 preferably comprises a brushless electric motor. Preferably, the electric motor 142 contains rare earth magnets surrounding a hollow core, in which the output shaft 145 resides. Of course, any suitable electric motor may be used. For example, the motor may be of brushed or brushless/sensorless design. Some considerations when selecting a suitable motor include toque rating (does the motor provide sufficient torque for a given application), power consumption (how much power is required to run the motor), size and weight (is the motor small and light enough to be easily handled), electromagnetic shielding (does the motor need to be electromagnetically shielded so as not to interfere with other surgical equipment or monitors), heat generation, and the like. One particularly preferred motor usable with our invention is manufactured by Carlsbad Magnetics, located in Carlsbad, Calif., (stator part number of 1060-2000 and a rotor part number of 1060-3000), and has a three phase winding, a peak torque of 25 ounces/in, a power of 41 watts at peak torque, a no load speed of 17,500 rotations/min, and a weight of 4.9 ounces.

Preferably the power supply 149 comprises a plurality of rechargeable NiCad cells of rapid charge design. More preferably, the power supply 149 comprises at least eight 1.2 volt NiCad cells. However, any other type of rechargeable battery may also be used, such as, for example, nickel metal hydride, lithium polymer, and the like. The voltage and storage requirements of the batteries will depend on considerations such as the power consumption of the motor and other electronic components, size, weight, and the like. While rechargeable batteries are one preferred power supply that can be used in our invention, we envision that numerous other types of portable power supplies may instead or additionally be used. By way of example, other suitable power supplies may include a single rechargeable battery or battery pack, one or more single-use batteries, fuel cells, and the like.

Charging contacts 147 are preferably provided on the power module 140 for recharging the batteries within the power module. Thus, in contrast to conventional power modules, there is no need to remove the batteries from the power module to charge them. Alternatively, charging could be accomplished by known inductive charging methods, without the need for direct contacts. If one or more rechargeable batteries or a battery pack are not used, or if the power module is configured for inductive charging, the charging contacts 147 may be omitted.

The electronic controller 146 preferably comprises a programmable electronic controller, which is capable of storing a plurality of software programs for monitoring and controlling functions of the power module. In one particularly preferred embodiment, the electronic controller 146 comprises a motor controller chip, such as the Three Phase Bridge N-Channel MOSFET Driver integrated circuit (part number HIP4086), manufactured by Intersil Americas Inc., headquartered in Milpitas, Calif., a microcontroller, such as the 28-pin, 8-bit CMOS FLASH Microcontroller with 10-bit A/D (part number PIC16F872), manufactured by Microchip Technology Inc., headquartered in Chandler, Ariz., and a programmable logic device, such as the CMOS Programmable Electrically Erasable Logic Device (part number 18CV8Z), manufactured by Integrated Circuit Technology Corp., located in Milpitas, Calif.

Because the power module 140 is installed into a blind receptacle 108 in the base of the hand unit 102, the electric motor 142 the output shaft 145 is preferably self-aligning, such that it automatically aligns with and engages a slotted drive shaft 120 of the hand unit 102. This self-aligning feature is accomplished by spring loading the output shaft 145, as shown in FIG. 2A. Thus, the output shaft 145 engages the slotted drive shaft 120 in the hand unit 102 or, if the output shaft 145 is not aligned with the slot in the drive shaft 120, it is depressed into the hollow cavity within the motor 142. When the motor 142 first rotates the output shaft 145 after installation of the power module 140, the output shaft 145 will become align with and be spring biased into engagement with the slot in the drive shaft 120 for power transmission. The engagement of the output shaft 145 with the drive shaft 120 transmits mechanical energy from the non-sterile power module 140 to the sterile hand unit 102.

As described above, the power module 140 is sealed in receptacle 108 by a lid 110, which is held closed by latch 138. In addition, the power module 140 preferably includes an annular ridge 148 that engages a deformable cylindrical coil spring retainer 128 in the hand unit 102 (FIG. 2A). The cylindrical coil spring 128 in its relaxed state has an inner circumference that is smaller than the outer circumference of the annular ridge 148 of the motor 142. Thus, during installation, a predetermined force must be applied to the power module 140 to expand the cylindrical coil spring 128 to allow the annular ridge 148 to be forced past. The cylindrical coil spring 128 then contracts into the valley formed behind the annular ridge 148 to prevent the non-sterile power module 140 from becoming accidentally detached from the sterile hand unit 102 and falling into the sterile surgical field. To remove the power module 140, a user has merely to apply a force sufficient to expand the cylindrical coil spring 128 to allow the annular ridge 148 pulled past.

The power module 140 also preferably includes a normally-open safety switch 143, which disconnects the electrical power supply 149 from the other electronic components until the power module 140 is installed in the hand unit 102. This prevents the power supply 149 from becoming discharged prior to use, as well as preventing inadvertent activation of the power module 140 outside the hand unit 102. The safety switch 143 closes to reconnect power to the other electronic components when it abuts an engagement contact 130 in the receptacle 108 of the hand unit 102 when the power module 140 is installed in the receptacle 108. The safety switch 143 can also be configured to prevent activation of the power module 140 while it is being installed in the hand unit 102, such as when the user is depressing the trigger 116 while installing the power module 140. In that case, the power module 140 will not run until the trigger 116 is completely released and then pressed again.

Once installed, the power module 140 can be controlled by actuation of the trigger 116 by a user. As described above, and as best shown in FIGS. 2 and 2A, actuation of the trigger is preferably communicated to the power module 140 via a series of rack and pinion gears, which in turn engages the speed control mechanism 144 of the power module 140. Preferably, the speed control mechanism 144 is a variable speed and variable direction control mechanism, and comprises one or more magnets that are movable relative to one or more Hall effect sensors. Engagement of the speed control mechanism 144 varies the proximity of the magnet(s) relative to the Hall sensor(s) and, consequently, the voltage(s) output from the sensor(s). An output of the speed control mechanism 144 is fed to the electronic controller 146. Based on the output from the speed control mechanism 144, the electronic controller 146 derives the desired speed and or direction of the motor 142, and outputs an appropriate drive signal to control the motor 142.

Preferably the electronic controller 146 provides variable speed and direction control of the motor. Alternatively, the electronic controller may be programmed to control the motor for on/off control with no variable speed control, to operate in an oscillate mode where the motor oscillates directions, to operate only in a forward direction, to operate only in a rearward direction, to have a selectable maximum speed setting (at full depression of the trigger 116), to have a selectable maximum torque setting (i.e., current limiting), or the like. Moreover, we envision that the electronic controller 146 could be programmed with a plurality of the foregoing control modes, and a user can switch between the programmed modes by, for example, toggling the trigger 116 a predetermined number of times, or by the provision of a switch or other user interface (not shown) on the power module 140 or hand unit 102. Provision of such switches and/or control methods would be readily apparent to one of ordinary skill in the art.

Still further, the power module 140 preferably includes one or more motor sensors (not shown) in communication with the electric motor 142, to monitor the speed, velocity, acceleration, and the like of the electric motor 142. The motor sensors are preferably embodied as one or more Hall effect sensors that measure magnetic fields generated by the motor. Of course, other types of conventional motor sensors can additionally, or alternatively, be used. Outputs of the motor sensors can be fed to the electronic controller 146 to provide information about the actual speed, velocity, and/or acceleration of the electric motor 142.

The power module 140 may also advantageously include other sensors, such as temperature sensors, current sensors, voltage sensors, counters, timers, and the like. The outputs of these sensors, counters, and timers may also be connected to the electronic controller 146, in order to provide information regarding the current state of the various electronic components of the power module 140.

In addition to providing electronic motor control, the electronic controller 146 preferably includes various known programs for monitoring and controlling the status and functions of the power module. This monitoring and control may be done independently by the electronic controller 146, or may be based on input from one or more of the previously described sensors, counters, timers, or the like. One such program that may be programmed in the electronic controller 146 monitors and controls electric current, and will regulate and/or stop the flow of electrical current, to prevent damage to sensitive electrical components and assemblies.

Another safety feature that can be programmed in the electronic controller 146 is stall detection. This program senses (based on the output from one or more of the motor sensors) whether the surgical apparatus has stalled and, if so, stops the flow of electrical current, again protecting the electronic components and assemblies.

The electronic controller 146 may also be programmed to create instant braking of the electric motor 142 when the user of the device releases to trigger 116 of the hand unit 102. This feature provides the surgeon with greater control over the device.

The motor controller may also be programmed to monitor the discharge of the battery cells, and will stop the discharge of the rechargeable batteries 149 when a minimum limit is reached, to prevent complete battery discharge.

Another software program that may be stored in the electronic controller 146 monitors the temperature of the electronic components, and shuts the system down if a predetermined maximum threshold temperature is reached. This program will prevent the electric motor 142 from being actuated again until a safe operating temperature is reached. This feature also protects the electronic components.

The electronic controller 146 may collect data such as the number of times the power module 140 is activated, whether the power module 140 has been subjected to sterilization temperatures, and the like.

The electronic controller 146 may also be programmed to have a “sleep mode,” which recognizes when the power module has been inactive for an extended period of time within the hand unit. The sleep mode will disconnect the power supply 149 from the other electronics to prevent total discharge of the battery cells or other power supply. The electronic controller 146 may be awakened by, for example, quickly pulling the trigger 116 twice in succession, removing and reinstalling of the power module 140 into the hand unit 102, or the like.

Method of Preparing

As noted above, the power module 140 of our invention avoids many of the problems in the prior art, because it need not be sterilized prior to use. Rather the non-sterile power module 140 is inserted and sealed within the receptacle 108 of the sterile hand unit 102. Insertion of the power module 140 without contaminating the sterile hand unit 102 is facilitated by the use of a transfer sleeve 190, such as the one shown in FIG. 9A. The transfer sleeve 190 fits over the opening in the receptacle 108 of the hand unit 102 to mask the hand unit 102 from contamination from the non-sterile power module during installation. The transfer sleeve 190 is then removed and either disposed of, or sterilized for reuse. The transfer sleeve 190 comprises a skirt 194 corresponding in shape to the opening in the receptacle 108. The skirt 194 sits on and covers the edges of the opening of the receptacle 108. An enlarged collar 192 of the transfer sleeve 190 projects up and out from the skirt 194, and acts as a funnel to guide the power source 140 into the receptacle 108 without contacting any part of the hand unit 102. A lid cover 196 extends from the transfer sleeve 190 to cover the lid 110 of the receptacle, and to prevent the lid 110 from swinging closed and contacting the non-sterile power module 140 during installation.

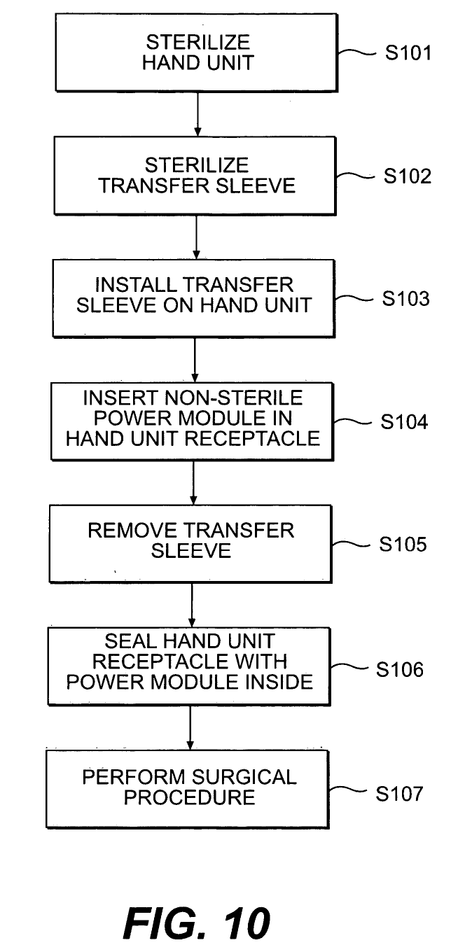

A method of preparing a surgical apparatus, using a power module according to our invention, for surgery, is described with reference to the flow chart of FIG. 10. First, in step S101, a user sterilizes the hand unit. Preferably sterilization is done using steam sterilization, as is conventionally done in the surgical field. However, the method of our invention applies equally to other types of sterilization, especially those involving the application of a liquid and/or heat. In step S102, the user provides a sterile transfer sleeve, either by sterilizing an existing transfer sleeve or by using a new, sterile transfer sleeve. Next, in step S103, the user installs the sterilized transfer sleeve on the opening of the receptacle of the hand unit. In step, S104, the user inserts the power module in the receptacle of the hand unit. Preferably, the power module comprises an electric motor, a power supply for supplying power to the electric motor and other electronic components, and a controller for controlling the power module. While the method of our invention is particularly useful with non-sterile power modules, sterile and sterilizable power modules may also be used to practice the method of our invention. Once the power module has been installed, the user removes the transfer sleeve in step S105. Then, in step S106, the user seals the power module inside the receptacle by closing the lid. The lid will automatically latch in the closed position. The next step S107 is to perform a surgical procedure using the apparatus. There is no need to first perform any further sterilization of the surgical apparatus. Using this method, the power module and the electronic components contained therein are not subjected to steam sterilization.

The embodiments discussed herein are representative of preferred embodiments of our invention and are provided for illustrative purposes only. Although specific structures, dimensions, components, etc., have been shown and described, such are not limiting. The various features and elements of the embodiments can be interchanged, rearranged, omitted, and/or combined in various different combinations to achieve a desired result. For example, we envision that the tool holders described herein may be adapted to be used with any conventional powered surgical apparatus hand unit, including pneumatic (e.g., powered by a rotary vane-type motor driven by compressed air, which may or may not be sterilizable) and corded hand units, as well as power module-powered hand units. The configuration and adaptation of such hand units for use with the tool holders described herein would be apparent to one of ordinary skill in the art.

Likewise, the configuration and adaptation of conventional hand units for use with the power modules described herein would be apparent to one of ordinary skill in the art

Moreover, while the various components of our invention have been shown and described as being of certain shapes and sizes, one of ordinary skill in the art would readily understand that the various components could be any shape and size desired for a given application. For example, while the hand units are described herein as being gun-shaped, hand units of other shapes and sizes may also be used. A few examples include hand units in the shape and size of a pen, smaller scope-mounted units, and the like.

Still further, while the drawings of our preferred embodiments show the tool holders holding sagittal and reciprocating saw blades, it would be apparent to one of ordinary skill in the art that the tool holders described herein could be used to hold any other type of tool having one of the hubs described herein. For example, we envision the tool holders described herein could be used to hold drills, driver bits, reams, wire driving or pinning attachments, and the like.

We claim:

1. A surgical apparatus comprising a tool holder, said tool holder comprising:
a substantially planar support surface;
a pair of spaced-apart sidewalls protruding from said support surface, each sidewall having at least one tab extending from said sidewall toward the other of said sidewalls;
a tool clamp, including a post protruding from said support surface with an enlarged head at a distal end thereof, said tool clamp being spaced substantially equally from each of said sidewalls; and
a lock button protruding from said support surface, said lock button being depressible toward said support surface and being spaced substantially equally from each of said sidewalls.

2. A surgical apparatus according to claim 1, said tool holder further comprising a boss protruding from said support surface and being spaced substantially equally from each of said sidewalls.

3. A surgical apparatus according to claim 2, said boss having a peripheral wall that is substantially perpendicular to said support surface and having a substantially wedge-shaped perimeter.

4. A surgical apparatus according to claim 1, said post of said tool clamp being slidable in an aperture in said support surface, and being biased toward said support surface to provide a clamping force for clamping a tool.

5. A surgical apparatus according to claim 1, said sidewalls being substantially parallel.

6. A surgical apparatus according to claim 1, each sidewall having a pair of spaced-apart tabs extending from a distal end of said sidewall toward the other of said sidewalls.

7. A surgical apparatus according to claim 1, further comprising a tool received in said tool holder, said tool comprising:
an elongated body;
a working surface disposed at a first end of said elongated body; and
a hub, by which the tool is held by said tool holder, disposed at a second end of said elongated body, said hub comprising:
a pair of lateral side surfaces, each said side surface having a notch formed therein; and
a slot having an opening at the second end of said elongated body, a terminus spaced from said opening toward the first end of said elongated body, and a pair of substantially parallel slot walls extending from said opening to said terminus, said slot having an expanded portion located between said opening and said terminus.

8. A surgical apparatus according to claim 7, said expanded portion comprising a pair of arcuate notches, one notch being formed on each of said slot walls.

9. A surgical apparatus according to claim 8, said hub of said tool further comprising a pair of stiffening surfaces, one stiffening surface extending between each of said slot walls and said second end of said elongated body, such that said stiffening surfaces are angled with respect to both said slot walls and said second end of said elongated body, said stiffening surfaces being adapted to engage said boss of said tool holder.

10. A surgical apparatus according to claim 1, said tool holder being coupled to and driven by a hand unit.

11. A surgical apparatus according to claim 10, said hand unit being powered by compressed air.

12. A surgical apparatus according to claim 10, further comprising a detachable power module received in said hand unit to provide power thereto, said power module comprising:
a housing;
an electric motor in said housing to provide mechanical energy to said hand unit;
an electrical power supply in said housing to provide electrical energy to said electric motor; and
an electronic controller in said housing for controlling said electric motor.

13. A tool comprising:
an elongated body;
a working surface disposed at a first end of said elongated body; and
a hub, by which the tool can be held, disposed at a second end of said elongated body, said hub comprising:
a pair of lateral side surfaces; and
a slot having an opening at the second end of said elongated body, a terminus spaced from said opening toward the first end of said elongated body, and a pair of substantially parallel slot walls extending from said opening to said terminus, said slot having an expanded portion located between said opening and said terminus.

14. A tool according to claim 13, each said side surface having a notch formed therein

15. A tool according to claim 13, said expanded portion comprising a pair of arcuate notches, one notch being formed on each of said slot walls.

16. A tool according to claim 13, said hub further comprising a pair of stiffening surfaces, one stiffening surface extending between each of said slot walls and said second end of said elongated body, such that said stiffening surfaces are angled with respect to both said slot walls and said second end of said elongated body, said stiffening surfaces being adapted to engage a boss of a tool holder.

17. A surgical apparatus comprising a tool holder, said tool holder comprising:
an output shaft having a driven end and a free end, said free end of said output shaft having a slot formed therein; and
a collet disposed coaxially with said output shaft and being rotatable relative thereto,
said collet having a first end adjacent to said free end of said output shaft, a second end spaced from said free end of said output shaft toward said driven end of said output shaft, and a collet wall extending between said first and second ends of said collet, and
said collet having a plurality of slots in said first end of said collet, a plurality of grooves in an inner surface of said collet wall, and a plurality of internal ridges protruding from the inner surface of said collet wall.

18. A surgical apparatus according to claim 17, said tool holder further comprising a torsion spring biasing said collet for rotation toward a lock position.

19. A surgical apparatus according to claim 17, wherein said plurality of grooves in the inner surface of said collet wall extend in paths having both circumferential and axial components.

20. A surgical apparatus according to claim 19, wherein said internal ridges protruding from the inner surface of said collet wall extend in paths substantially parallel to those of said plurality of grooves in the inner surface of said collet wall.

21. A surgical apparatus according to claim 17, said tool holder further comprising a section ring disposed in an annular groove formed in an outer surface of said output shaft.

22. A surgical apparatus according to claim 17, further comprising a tool received in said tool holder, said tool comprising:
an elongated body having a first end, a second end, and two lateral edges;
a working surface disposed along at least a portion of one lateral edge near the first end of said elongated body; and
a hub, by which the tool is held by said tool holder, disposed at the second end of said elongated body, said hub comprising:
a middle portion having a first width;
a pair of lateral recesses formed in said hub adjacent to said middle portion in the direction of the first end of said elongated body;
a pair of tangs protruding laterally from said hub adjacent to said lateral recesses in the direction of the first end of said elongated body; and
a narrow end portion, having a width less than the first width, adjacent to said middle portion at an end of said hub opposite the working surface.

23. A surgical apparatus according to claim 17, said tool holder being coupled to and driven by a hand unit.

24. A surgical apparatus according to claim 23, said hand unit being powered by compressed air.

25. A surgical apparatus according to claim 23, further comprising a detachable power module received in said hand unit to provide power thereto, said power module comprising:
a housing;
an electric motor in said housing to provide mechanical energy to said hand unit;
an electrical power supply in said housing to provide electrical energy to said electric motor; and
an electronic controller in said housing for controlling said electric motor.

26. A tool comprising:
an elongated body having a first end, a second end, and two lateral edges;
a working surface disposed along at least a portion of one lateral edge near the first end of said elongated body; and
a hub, by which the tool can be held, disposed at the second end of said elongated body, said hub comprising:
a middle portion having a first width;
a pair of lateral recesses formed in said hub adjacent to said middle portion in the direction of the first end of said elongated body;
a pair of tangs protruding laterally from said hub adjacent to said lateral recesses in the direction of the first end of said elongated body; and
a narrow end portion, having a width less than the first width, adjacent to said middle portion at an end of said hub opposite the working surface.
